(19) United States
(12) Patent Application Publication
(10) Pub. No.: US 2010/0076444 A1
(43) Pub. Date: Mar. 25, 2010

(54) Bone distraction device having a quick release disengagement mechanism

(76) Inventors: Hienz Staehler, Muhlheim (DE); Thomas Johnston, Jacksonville, FL (US)

Correspondence Address: ROGERS TOWERS, P.A., 1301 RIVERPLACE BOULEVARD, SUITE 1500, JACKSONVILLE, FL, 32207, US

(21) Appl. No.: 12/284,679

(22) Filed: Sep. 24, 2008

(51) Int. Cl. A61B 17/58

(52) U.S. Cl. 606/90

(57) ABSTRACT

A bone distraction device having a ratchet and pawl mechanism such that incremental rotation is allowed in one direction and prevented in the other, and further having a quick release mechanism such that the ratchet and pawl mechanism can be released to allow free rotation in both directions.

### BACKGROUND OF THE INVENTION

This invention relates generally to the field of medical distraction devices in the field of bone fixation, repair and regeneration, and more particularly relates to such devices and methods utilized in mandibular and maxillary craniofacial repair, facial reconstruction, and treatment for congenital, developmental and traumatic, defects.

It is often desirable or necessary to effect reconstruction or repair of the craniofacial bones defining the face of a person, such as the midface, the mandible and/or the maxilla. This need may arise from congenital conditions, developmental disorders or trauma. In many circumstances the abnormalities are corrected by first dividing a bone through osteotomy, i.e., cutting or fracturing a bone to create two segments separated by a gap or space. In some instances the bone segments will be shifted into the proper orientation and alignment, and then fixed in place relative to each other until bone growth across the gap results in the formation of a unitary bone member. In other instances it is necessary to lengthen the original bone member, in which case devices known as distractors are utilized. A distractor is a device that has affixation means, such as bone plates, that are joined to each of the bone segments on opposite sides of the osteotomy using mechanical fasteners such as bone screws. The device further includes distraction means that allows the distance between the bone plates to be slowly increased over time, thereby allowing new bone growth to occur between the bone segments. The distraction means may comprise for example a threaded rod contained within a framework or elongated housing, the rod being in connection with one or two translatable shoe or mounting members to which are connected a proximal bone plate and a distal bone plate, whereby the bone plates affixed on either side of the osteotomy gap to the bone to be extended can be gradually separated in a controlled and precise manner by rotating the threaded rod to separate the plate mounting members. The new bone growth increases in dimension until the proper bone length is achieved, at which time the distraction process is halted and the distractor is removed. U.S. Pat. No. 6,752,808 issued to Schumacher illustrates and describes a distraction device of the type referenced above.

A typical example of this procedure is when the mandible or jawbone fails to fully develop in the anterior-posterior direction, a condition known as mandibular hypoplasia, which is manifested as a severely fore-shortened chin. To correct this anomaly, osteotomies are performed on each side of the mandible and a pair of distraction devices are affixed to the mandible. Extension of the distractors is performed in unison to lengthen the mandible until the desired position of the mandible relative to the midface is achieved. After sufficient regeneration and healing, the distractors are removed.

For another example, it is often desired to advance the midface or maxillary region relative to the jaw and skull to correct for maxillary hypoplasia, where the upper lip and/or nose are depressed relative to the remainder of the face structure. In this case the osteotomy may be performed across the maxilla to the nasal cavity, and a pair of distractors are affixed across the osteotomy gap, or an external distraction apparatus is mounted to the skull with affixed means to distract the anterior maxillary segment. Gradual extension of the distractors in unison advances the anterior maxillary segment relative to the posterior maxillary segments while bone regeneration fills in the osteotomy gap. When the proper position is achieved, distraction is halted. After sufficient regeneration and healing, the distractors are removed.

It is desirable that the distraction be performed in a controlled or incremental manner, whereby the advancement distance can be accurately accounted for, and it is further desirable that the apparatus allow for rotation in only one direction to insure that the bone plates are separated rather than contracted. This may be accomplished by utilizing a ratchet and pawl mechanism or having mating teeth, such that rotation of the drive head joined to the threaded rod and relative movement of the bone plates can only occur in the distraction direction. Examples of distraction devices utilizing ratchet and pawl mechanisms or mating teeth are seen in U.S. Pat. No. 5,681,309 to Ross, Jr. et al., U.S. Pat. No. 5,704,938 to Stawhlin et al., U.S. Pat. No. 6,033,412 to Losken et al., and U.S. Pat. No. 6,277,124 to Haag.

One problem with such unidirectional distraction devices is that it may be desirable or required to contract the bone plates by rotating the threaded drive rod in the direction opposite to the distraction direction. In certain circumstances, it may be necessary to allow for free rotation of the threaded drive rod rather than incremental rotation. It may also be necessary in certain circumstances to release the threaded drive rod quickly. Therefore, it is desirable and an object of this invention to provide a distraction device having a ratchet and pawl means that allows for incremental rotation of the threaded drive rod in the distraction direction, wherein a quick release disengagement means is provided such that the ratchet and pawl mechanism can be disengaged and the drive rod rotated in the opposite direction as needed.

### SUMMARY OF THE INVENTION

The invention is a bone distractor, and is especially drawn to a bone distractor used in mandibular and maxillary craniofacial repair, reconstruction and treatment that enables a surgeon to lengthen the maxilla or the mandible. In a representative illustration, osteotomies are provided on the left and right sides of the mandible or the maxilla (or both), such that the anterior portion of the mandible or the maxilla is divided from the posterior portion. This allows the anterior portions to be repositioned in a more desirable orientation.

The bone distractor of the invention comprises means for attachment of the device to a bone on opposite sides of an osteotomy gap, such as by a pair of bone plate members affixed by bone screws or similar bone attachment members known in the art. The bone distractor further comprises distraction means to separate the bone plates, such that the bone portions to which the device is attached are gradually separated as well, such as by a threaded rod retained within a framework or housing, with the bone plate members mounted on one or two translatable mounting members in mechanical communication with the threaded rod, such that rotation of the threaded rod results in relative movement of one or both mounting members such that the bone plates move apart. The bone distractor device further comprises a ratchet and pawl mechanism whereby rotation of the threaded rod is allowed in only one direction while the pawl is engaged, the pawl serving to secure the threaded rod in incremental positions relative to the framework or housing, and further comprising disengagement means whereby the pawl can be quickly and easily released such that free rotation of the threaded rod in either direction is possible.

### BRIEF DESCRIPTION OF THE DRAWINGS

FIG. 1 is a plan view of an illustrative embodiment of the unidirectional distraction device having a quick release disengagement mechanism.

FIG. 2 is a partial view of the device of FIG. 1, better illustrating the quick release disengagement mechanism.

### DETAILED DESCRIPTION OF THE INVENTION

With reference to the drawings, the invention will now be described in detail with regard for the best mode and the preferred embodiment.

The invention is a bone distractor, and is especially drawn to a bone distractor used in mandibular and maxillary craniofacial repair, reconstruction and treatment. In a representative procedure, osteotomies are provided on the left and right sides of the mandible or the maxilla, such that the anterior portion of the bone is divided from the posterior portion of the bone. A pair of distractor devices are utilized, one for each side of the mandible or maxilla. The distractor device is affixed to the mandible or maxilla spanning the osteotomy gap. Distraction is accomplished slowly to allow bone regeneration to occur across the osteotomy gap. When the bones are properly lengthened, the device is removed.

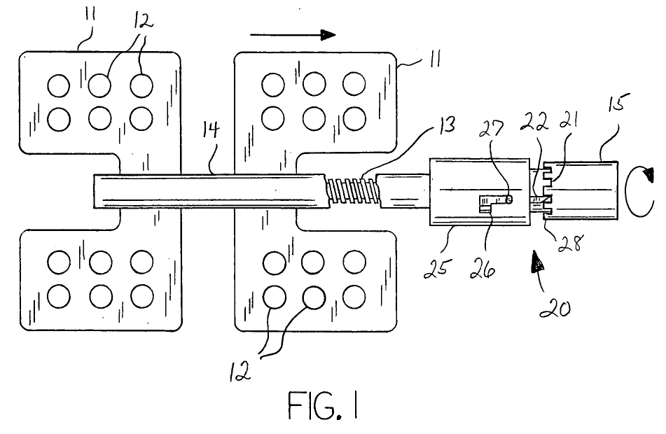

FIG. 1 illustrates a representative embodiment of the distraction device comprising distraction means for separating a pair of bone portions divided by an osteotomy, the distraction device comprising means for affixing the device to bone on either side of the osteotomy gap, such as for example a pair of bone plate members 11, the bone plate members 11 having apertures 12 to receive mechanical fastener means such as bone screws, as is well known in the art. The distraction means comprises a threaded rod 13 received within a framework or elongated rod housing 14 in a manner whereby the threaded rod 13 may be rotated by turning a drive head member 15. The bone plate members 11 are mounted to the threaded rod 13 in a manner whereby rotation of the threaded rod 13 causes the bone plates to separate or contract relative to each other, such as for example by utilizing an internally threaded mounting member or shoe (not shown) on one of the bone plate members 11 that receive the threaded rod 13, the mounting member being able to move longitudinally along the housing 14 when the threaded rod 13 is rotated. Such mechanisms are well known in the art. Typically, the drive head member 15 is provided with a drive tool receiving bore or slot, such that rotation of the threaded rod 13 is achieved by inserting the drive tool, such as for example a hexagonal shaft, into the bore and rotating the drive tool.

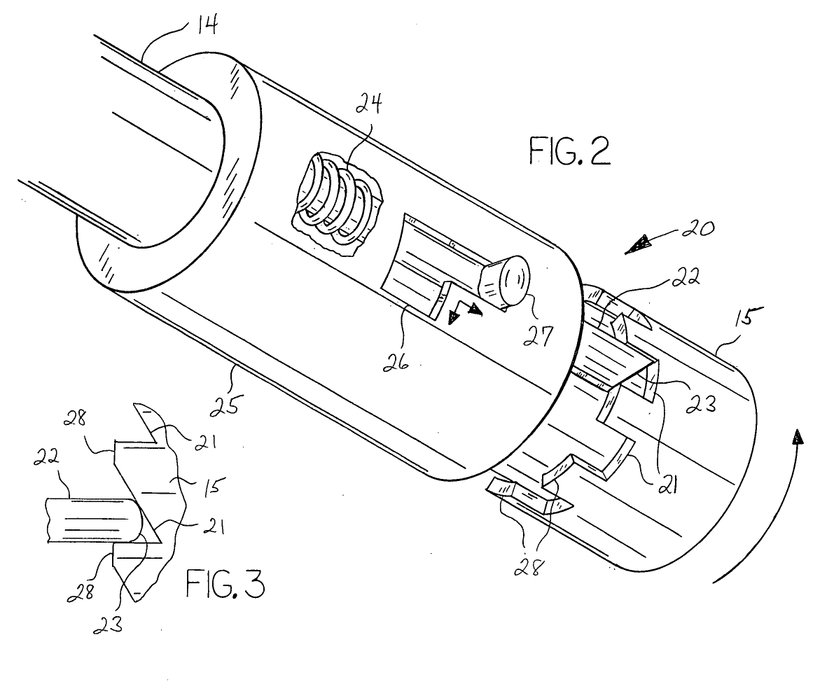

The distraction device further comprises incremental indexing means for allowing rotation of the drive head 15 and threaded rod 13 in a single direction via a ratchet and pawl assembly 20 that interacts with ratchet teeth 28 and notches 21 provided annularly on the drive head member 15. The pawl member 22 is provided with a curved or angled end 23 and is biased outwardly against the ratchet notches 21 by a biasing member 24. The pawl 22 is retained within a pawl housing 25 mounted on the rod housing 14. In this manner, the drive head 15 can be rotated in one direction, with the rotation resulting in the depression of the biasing member 24 into the pawl housing 25 because of the angled or curved end 23. When the drive head 15 brings the next notch 21 in alignment with the pawl 22, the biasing member 24 forces the pawl 22 outward, locking the drive head 15 in position until further rotation force is applied because the extended portion of the pawl end 23 opposite from the angle or curve abuts the side of the teeth 28, precluding rotation in the opposite direction. This structure results in separation of the bone plate members 11 by a defined distance with each turn. In an alternative embodiment, as shown in FIG. 3, the notches 21 may be provided with a curved or angled wall and the pawl end 23 presented as generally rectangular or cylindrical, in known manner, such that as before the drive head 15 can be rotated in only one direction when the pawl 22 is engaged, which in this figure is shown opposite to the direction of FIG. 2.

The distraction device further comprises disengagement means for releasing the ratchet and pawl mechanism such that the drive head 15 can be freely rotated in either direction. The disengagement means comprises an L-shaped slot 26 disposed in the pawl housing 25 and a radially extending post member 27 that is mounted on the pawl 22 and extends through the L-shaped slot 26. To disengage the pawl 22 from the drive head 15, the post member 27 is moved longitudinally within the slot 26 to depress the biasing member 24 and retract the pawl a sufficient distance such that it does not contact the notches 21. The post member 27 is then moved circumferentially preclude it from being advanced outwardly by the biasing member 24. In this disengaged state, the drive head 15 can now be rotated in either direction such that the bone plate members 11 can be either contracted or distracted. To engage the pawl 22, the post member 27 is simple moved back into the position where the biasing member 24 can push it and the pawl longitudinally within the slot 26, thereby extending the pawl end 23 into one of the notches 21 of the drive head 15. Alternatively, the slot 26 can be linear, such that the post member 27 must be held in the retracted position to allow for free rotation of the drive head 15 in either direction.

It is contemplated that equivalents and substitutions for elements set forth above may be obvious to those of ordinary skill in the art, and therefore the true scope and definition of the invention is to be as set forth in the following claims.

We claim:

1. A bone distraction device comprising
a pair of bone plate members;
a threaded rod retained within a rod housing, said threaded rod interacting with said bone plate members whereby rotation of said threaded rod within said rod housing in a distraction direction results in relative separation or contraction of said bone plate members depending upon the direction of rotation;
a drive head connected to said threaded rod, whereby rotation of said drive head rotates said threaded rod, said drive head having teeth and notches disposed annularly thereon;
a pawl housing positioned on said rod housing, said pawl housing retaining a pawl biased outwardly into one of said notches on said drive head to define an engaged position, said pawl and said notches configured such that said drive head is rotatable only in said distraction direction when said pawl is in said engaged position;
a slot disposed in said pawl housing and a post member connected to said pawl and extending through said slot, whereby said pawl is retracted away from said drive head notches teeth by moving said post member longitudinally within said slot to define a disengaged position, such that rotation of said drive head is allowable in either direction when said pawl is in said disengaged position.

2. The device of claim 1, wherein said pawl comprises a pawl end and wherein said pawl end is angled or curved.

3. The device of claim 1, wherein said ratchet notches comprise an angled or curved wall.

4. The device of claim 1, wherein said slot is L-shaped.

5. The device of claim 1, further comprising a compressible spring member connected to said pawl, such that said spring member maintains said pawl in said engaged position.

6. A bone distraction device, said device comprising:
distraction means for separating a pair of bone portions divided by an osteotomy, said distraction means comprising a rotating drive head connected to a rotating threaded rod;
incremental indexing means for allowing incremental rotation of said drive head and said threaded rod in a single distraction direction, said incremental indexing means comprising a ratchet and pawl assembly;
said ratchet and pawl assembly comprising teeth and notches annularly disposed on said drive head and a retractable pawl member disposed in cooperation with said teeth and said notches, said pawl member having an engaged position abutting said teeth and said notches allowing incremental rotation of said drive head only in said distraction direction, and said pawl member having a disengaged position separated from said teeth and said notches allowing free rotation of said drive head in either direction.

7. The device of claim 6, wherein said pawl member comprises a pawl end and wherein said pawl end is angled or curved.

8. The device of claim 6, wherein said ratchet notches comprise an angled or curved wall.

9. The device of claim 6, said ratchet and pawl assembly further comprising a pawl housing, a slot disposed on said pawl housing and a post member connected to said pawl member and extending through said slot, whereby said pawl member is moved from said engaged position to said disengaged position by moving said post member within said slot.

10. The device of claim 9, wherein said slot is L-shaped.

11. The device of claim 6, further comprising a compressible spring member connected to said pawl member, such that said spring member maintains said pawl member in said engaged position.

12. The device of claim 9, further comprising a compressible spring member connected to said pawl member, such that said spring member maintains said pawl member in said engaged position.

13. The device of claim 10, further comprising a compressible spring member connected to said pawl member, such that said spring member maintains said pawl member in said engaged position.

14. A bone distraction device, said device comprising:
distraction means for separating a pair of bone portions divided by an osteotomy, said distraction means comprising a rotating drive head connected to a rotating threaded rod;
incremental indexing means for allowing incremental rotation of said drive head and said threaded rod in a single distraction direction, said incremental indexing means comprising a ratchet and pawl assembly;
said ratchet and pawl assembly comprising teeth and notches annularly disposed on said drive head and a retractable pawl member disposed within a pawl housing and acting in combination with said teeth and said notches, said pawl member having an engaged position abutting said teeth and said notches allowing incremental rotation of said drive head in said distraction direction only, and said pawl member having a disengaged position separated from said teeth and said notches allowing free rotation of said drive head in either direction;
disengagement means for releasing said pawl member such that said drive head and said threaded rod are freely rotatable in both directions, said disengagement means comprising a slot disposed on said pawl housing, a post member connected to said pawl member and extending through said slot, and a compressible spring member connected to said pawl member, such that said spring member biases said pawl member in said engaged position, whereby said pawl member is moved from said engaged position to said disengaged position by moving said post member within said slot to compress said spring member.

15. The device of claim 14, wherein said pawl member comprises a pawl end and wherein said pawl end is angled or curved.

16. The device of claim 14, wherein said ratchet notches comprise an angled or curved wall.

17. The device of claim 14, wherein said slot is L-shaped.
